(19) United States
(12) Patent Application Publication
(10) Pub. No.: US 2010/0076081 A1
(43) Pub. Date: Mar. 25, 2010

(54) METHOD FOR PRODUCING SALT OF 4-SULFINYLAMINO-1-CYCLOHEXANECARBOXYLIC ACID

(76) Inventor: Sohei Omura, Hyogo (JP)

Correspondence Address: BIRCH STEWART KOLASCH & BIRCH, PO BOX 747, FALLS CHURCH, VA, 22040-0747, US

(21) Appl. No.: 12/443,330

(22) PCT Filed: Sep. 26, 2007
(86) PCT No.: PCT/JP2007/068607

§ 371 Date: Mar. 27, 2009

(30) Foreign Application Priority Data
Sep. 28, 2006 (JP) 2006-263682

(51) Int. Cl. A61K 31/19; C07C 311/07

(52) U.S. Cl. 514/557; 562/507; 564/80

(57) ABSTRACT

Disclosed is a salt of trans-4-sulfinylamino-1-cyclohexanecarboxylic acid. Also disclosed is a method for producing a salt of trans-4-sulfinylamino-1-cyclohexanecarboxylic acid.

### FIELD OF THE INVENTION

This invention relates to a method for producing a salt of trans-4-sulfinylamino-1-cyclohexanecarboxylic acid.

### BACKGROUND ART

A salt of trans-4-sulfinylamino-1-cyclohexanecarboxylic acid is a useful compound as a starting material or an intermediate for synthesizing pharmaceuticals, which can be utilized, for example, as an intermediate for synthesizing a compound having an NPY Y5 receptor antagonistic activity described in Patent Document 1.

Patent Document 1 discloses a process for the preparation of 4-(2-methylpropane-2-sulfonylamino-1-cyclohexanecarboxylic acid which comprises subjecting 4-amino-2-cyclohexanecarboxylic acid methyl ester and t-butylsulfinyl chloride to the coupling reaction in a dichloromethane solvent, oxidizing the obtained compound, and then hydrolysis thereof. Industrial application of the process is difficult because it requires the use of dichloromethane, which is limited, and the isolation of the product by chromatography.

Patent Document 2 discloses a process for the preparation of trans-4-(2-methylpropane-2-sulfonylamino-1-cyclohexanecarboxylic acid which comprises subjecting cis-4-amino-1-cyclohexanecarboxylic acid methyl ester and t-butylsulfinyl chloride to the coupling reaction in an ethyl acetate solvent, oxidation reaction, transformation to trans isomer, and then hydrolysis. This process was far from a high-yielding preparation process as the yield from cis-4-amino-1-cyclohexanecarboxylic acid to trans-4-(2-methylpropane-2-sulfonylamino) cyclohexanecarboxylic acid is 70% or less even if the loss in transformation to trans isomer was not counted.

Patent Document 3 discloses a process for the preparation of trans-4-sulfonylamino-1-cyclohexanecarboxylic acid which comprises oxidation of trans-4-sulfinylamino-1-cyclohexanecarboxylic ester to give trans-4-sulfonylamino-1-cyclohexanecarboxylic ester, and hydrolysis thereof.

### [Patent Document 1] WO01/37826

### [Patent Document 2] WO2003/076374

### [Patent Document 3] JP2005-255630

### DISCLOSURE OF INVENTION

### Problems to be Solved by the Invention

The present invention provides efficient processes for the preparation of trans-4-sulfonylamino-1-cyclohexanecarboxylic acid which is useful as a starting material or an intermediate for synthesizing pharmaceuticals.

### Means for Solving the Problem

In contrast to the process disclosed in Patent Document 3, there is an efficient process for the preparation of trans-4-sulfonylamino-1-cyclohexanecarboxylic acid disclosed in PCT/JP2006/306616, comprising hydrolysis of trans-4-sulfinylamino-1-cyclohexanecarboxylic ester to give trans-4-sulfinylamino-1-cyclohexanecarboxylic acid and then oxidation thereof.

The present inventor found that an acidity or alkalinity of trans-4-sulfinylamino-1-cyclohexanecarboxylic acid solution is important when trans-4-sulfinylamino-1-cyclohexanecarboxylic acid is oxidized after hydrolysis of trans-4-sulfinylamino-1-cyclohexanecarboxylic ester for preparing trans-4-sulfinylamino-1-cyclohexanecarboxylic acid efficiently in the above-mentioned process.

The present inventor found that the pH between 6 and 11 is preferred as an acidity or alkalinity of the solution for oxidation reaction of the solution containing trans-4-sulfinylamino-1-cyclohexanecarboxylic acid or its salt.

The present inventor also found that a highly purified trans-4-sulfonylamino-1-cyclohexanecarboxylic acid can be obtained by isolating a salt of trans-4-sulfinylamino-1-cyclohexanecarboxylic acid and oxidizing its aqueous solution.

The present invention includes:

(1) A salt or its solvate, of a compound represented by the formula (I):

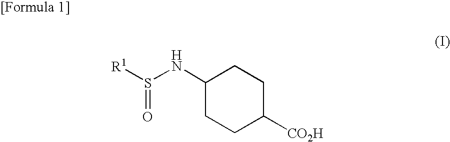

wherein R¹ is optionally substituted lower alkyl, optionally substituted cycloalkyl or optionally substituted aryl.
(2) The salt or its solvate, of the compound according to the above (1), wherein the salt is an inorganic salt.
(3) The salt or its solvate, of the compound according to the above (1) or (2), wherein the salt is selected from a group consisting of sodium salt, lithium salt, potassium salt, magnesium salt, calcium salt, barium salt and cesium salt.
(4) The salt or its solvate, of the compound according to the above (1), wherein the salt is an organic salt.
(5) The salt or its solvate, of the compound according to the above (4), wherein the salt is selected from a group consisting of pyrrolidine salt, diisopropylamine salt, tert-butylamine salt, isopropylamine salt, diisopropylethylamine salt, piperazine salt, piperidine salt, morpholine salt and N-methyl morpholine salt.
(6) An aqueous solution of pH between 6 and 11 containing a compound represented by the formula (I)

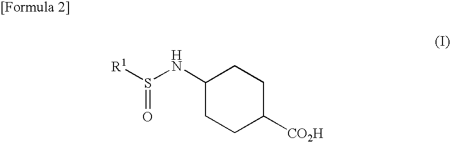

wherein R¹ has the same meaning as defined in the above (1),
or its salt.
(7) A process for the preparation of an aqueous solution of pH between 6 and 11 containing a compound represented by the formula (I):

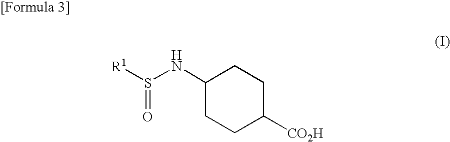

wherein R¹ has the same meaning as defined in the above (1), or its salt,
which comprises neutralizing with an acid an aqueous solution containing a salt of the compound represented by the formula (I).
(8) A process for the preparation of Compound (II):

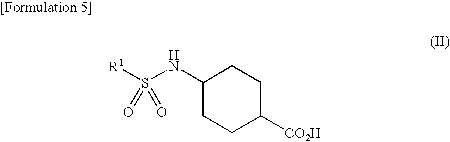

wherein R¹ has the same meaning as defined in the above (1),
which comprises oxidizing an aqueous solution of pH between 6 and 11 containing a compound represented by the formula (I):

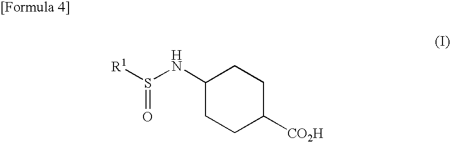

wherein R¹ has the same meaning as defined in the above (1),
or its salt.
(9) A process for the preparation of a salt or its solvate, of a compound represented by the formula (I):

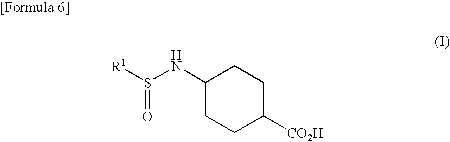

wherein R¹ has the same meaning as defined in the above (1),
which comprises dissolving or suspending a compound represented by the formula (I) in water and/or organic solvent and adding a base thereto.
(10) A process for the preparation of a compound represented by the formula (IV):

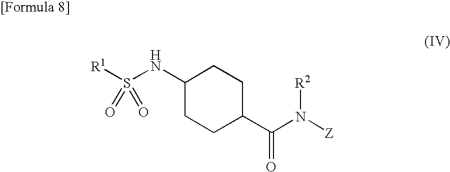

wherein R¹ has the same meaning as defined in the above (1), R² is hydrogen or lower alkyl and Z is optionally substituted lower alkyl, optionally substituted lower alkenyl, optionally substituted amino, optionally substituted lower alkoxy, optionally substituted cyclic hydrocarbon group or optionally substituted heterocyclic group, its pharmaceutically acceptable salt or solvate thereof,
which comprises reacting Compound (II) derived according to any one of the above (1) to (9):

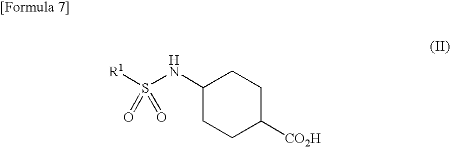

wherein R¹ has the same meaning as defined above,
with Compound represented by the formula: R²NH—Z (III)
wherein.R² and Z have the same meaning as defined above.
(11) An aqueous solution of the pH between 6.6 and 7.4 containing a compound represented by the formula (I):

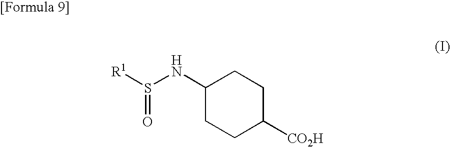

wherein R¹ has the same meaning as defined in the above (1),
or its salt.
(12) A process for the preparation of an aqueous solution of the pH between 6.6 and 7.4 containing a compound represented by the formula (I):

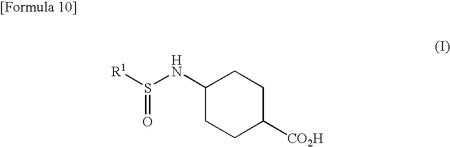

wherein R¹ has the same meaning as defined in the above (1),
or its salt,
which comprises neutralizing with an acid an aqueous solution containing a salt of the compound represented by the formula (I).
(13) A process for the preparation of a compound represented by the formula (II):

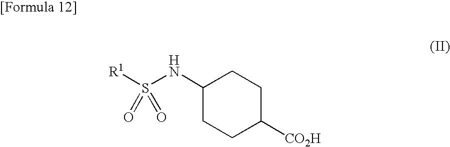

wherein R¹ has the same meaning as defined in the above (1),
which comprises oxidizing an aqueous solution of the pH between 6.6 and 7.4 containing a compound represented by the formula (I):

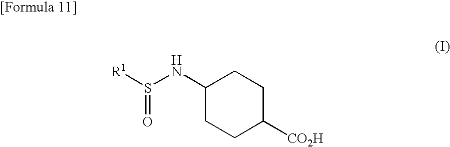

wherein R¹ has the same meaning as defined in the above (1),
or its salt.

### EFFECT OF THE INVENTION

As evidenced by the Examples described below, a salt of Compound (I) of the present invention is a useful compound as a starting material or an intermediate for synthesizing pharmaceuticals and the like. A new process for the preparation of the salt of Compound (I) can be utilized as an industrial preparation because of the safety and the high yield.

### BEST MODE FOR CARRYING OUT THE INVENTION

In the present description, “lower alkyl” includes C1 to C10, preferably C1 to C6, and more preferably C1 to C3 straight or branched alkyl group. Examples include methyl, ethyl, n-propyl, isopropyl, n-butyl, isobutyl, sec-butyl, t-butyl, n-pentyl, isopentyl, neopentyl, hexyl, isohexyl, n-heptyl, isoheptyl, n-octyl, isooctyl, n-nonyl, n-decyl and the like.

“Lower alkyl” of R¹ is preferably ethyl, isopropyl or t-butyl.

The lower alkyl part of “aryl lower alkyl”, “halogeno lower alkyl” or “hydroxy lower alkyl” is the same as the above “lower alkyl”.

Examples of the substituents for “optionally substituted lower alkyl” of Z include (1) halogen; (2) cyano; and (3) the following groups (i) to (xvi): (i) hydroxy, (ii) lower alkoxy, mercapto, (iv) lower alkylthio, (v) acyl, (vi) acyloxy, (vii) carboxy, (viii) lower alkoxycarbonyl, (ix) imino, (x) carbamoyl, (xi) thiocarbamoyl, (xii) lower alkylcarbamoyl, (xiii) lower alkylthiocarbamoyl, (xiv) amino, (xv) lower alkylamino or (xvi) heterocyclylcarbonyl, which may be optionally substituted with at least one of groups selected from Substituent Group β (beta) defined below.

Examples of the substituents for “optionally substituted lower alkyl” include at least one of groups selected from Substituent Group β (beta) defined below.

“Lower alkenyl” includes C2 to C10, preferably C2 to C8 and more preferably C3 to C6 straight or branched alkenyl having at least one double bond at any position(s). Examples are vinyl, propenyl, isopropenyl, butenyl, isobutenyl, prenyl, butadienyl, pentenyl, isopentenyl, pentadienyl, hexenyl, isohexenyl, hexadienyl, heptenyl, octenyl, nonenyl, decenyl and the like.

Examples of the substituents for “optionally substituted lower alkenyl” include halogen, lower alkoxy, lower alkenyl, amino, lower alkylamino, lower alkoxycarbonylamino, lower alkylthio, acyl, carboxy, lower alkoxycarbonyl, carbamoyl, cyano, cycloalkyl, phenyl, lower alkylphenyl, lower alkoxyphenyl, naphthyl, heterocyclyl and/or the like.

Examples of the substituents for “optionally substituted amino” include Substituent Group β (beta) defined below, optionally substituted benzoyl and/or optionally substituted heterocyclylcarbonyl (wherein the substituent is hydroxy, lower alkyl, lower alkoxy and/or lower alkylthio).

The lower alkyl part of “lower alkoxy”, “lower alkylthio”, “lower alkylcarbamoyl”, “lower alkylthiocarbamoyl”, “lower alkylamino”, “di-lower alkylamino”, “lower alkylsulfinyl”, “lower alkylsulfonyl”, “lower alkylsulfamoyl”, “lower alkoxycarbonyl”, “lower alkoxy lower alkyl”, “hydroxy lower alkyl”, “lower alkoxycarbonylamino”, “lower alkylphenyl”, “lower alkoxyphenyl”, “halogeno lower alkyl”, “phenyl lower alkoxy” or “phenyl lower alkylthio” is the same as the above “lower alkyl”.

Examples of the substituents for “optionally substituted lower alkoxy” include at least one of groups selected from Substituent Group β (beta) defined below. Preferred is phenyl, lower alkylphenyl, lower alkoxyphenyl, naphthyl or heterocyclyl.

“Acyl” includes (1) C1 to C10, more preferably C1 to C6 and most preferably C1 to C4 straight or branched alkylcarbonyl or alkenylcarbonyl, (2) C4 to C9 and preferably C4 to C7 cycloalkylcarbonyl and (3) C7 to C11 arylcarbonyl. Examples include formyl, acetyl, propionyl, butyryl, isobutyryl, valeryl, pivaloyl, hexanoyl, acryloyl, propioloyl, methacryloyl, crotonoyl, cyclopropylcarbonyl, cyclohexylcarbonyl, cyclooctylcarbonyl, benzoyl and the like.

The acyl part in “acyloxy” is the same as the above.

The protecting group of “optionally protected hydroxy” or “optionally protected hydroxy lower alkyl” includes all hydroxy protecting groups which are usually used. Examples include acyl (e.g., acetyl, trichloroacetyl and benzoyl), lower alkoxycarbonyl (e.g., t-butoxycarbonyl), lower alkylsulfonyl (e.g., methanesulfonyl), lower alkoxy lower alkyl (e.g., methoxymethyl), trialkylsilyl (e.g., t-butyldimethylsilyl) and the like.

“Halogen” includes fluorine, chlorine, bromine and iodine. Especially preferred is fluorine or chlorine.

The halogen part of “halogenophenyl” or “halogeno lower alkyl” is the same as the above “halogen”.

“Alkylenedioxy” includes methylenedioxy, ethylenedioxy, trimethylenedioxy, tetramethylenedioxy, pentamethylenedioxy and hexamethylenedioxy. Preferred is methylenedioxy or ethylenedioxy.

“Cyclic hydrocarbon group” includes “cycloalkyl”, “cycloalkenyl”, “bicycloalkyl” and “aryl”.

“Cycloalkyl” includes C3 to C8 and preferably C5 to C6 cyclic alkyl. Examples are cyclopropyl, cyclobutyl, cyclopentyl, cyclohexyl, cycloheptyl, cyclooctyl and the like.

Examples of the substituents for “optionally substituted cycloalkyl” include at least one of groups selected from Substituent Group β (beta) defined below.

“Cycloalkenyl” includes the above cycloalkyl with at least one double bond at any position(s) in the ring. Examples include cyclopropenyl, cyclobutenyl, cyclopentenyl, cyclohexenyl, cyclohexadienyl and the like.

“Bicycloalkyl” includes a group which is derived by excluding one hydrogen atom from a C5 to C8 aliphatic cycle which consists of two rings that share two or more atoms. Examples include bicyclo[2.1.0]pentyl, bicyclo[2.2.1]heptyl, bicyclo[2.2.2]octyl, bicyclo[3.2.1]octyl and the like.

“Aryl” includes monocyclic or polycyclic aromatic carbocyclyl, and examples include phenyl, naphtyl, anthryl, phenanthryl and the like. It also includes aryl which is fused with another non-aromatic cyclic hydrocarbon group, and examples include indanyl, indenyl, biphenylyl, acenaphthyl, tetrahydronaphthyl, fluorenyl and the like. Especially preferred is phenyl.

Examples of the substituents for “optionally substituted cyclic hydrocarbon group” include at least one of groups selected from Substituent Group α (alpha) and β (beta) defined below. The “cyclic hydrocarbon group” may be substituted at any position(s).

Examples of the substituents for “optionally substituted aryl” of R¹ include at least one of groups selected from the group consisting of halogen, optionally protected hydroxy, mercapto, lower alkyl, halogeno lower alkyl, hydroxy lower alkyl, lower alkoxy, lower alkenyl, di-lower alkylamino, lower alkylthio, acyl, carboxy, lower alkoxycarbonyl, carbamoyl, cyano, cycloalkyl, phenyl, phenoxy, lower alkyl phenyl, lower alkoxy phenyl, halogenophenyl, naphthyl and heterocyclyl.

Examples of the substituents for the other “optionally substituted aryl” include at least one of groups selected from Substituent Group β (beta) defined below.

The cycloalkyl part of “cycloalkylcarbamoyl”, “cycloalkylsulfamoyl” or “cycloalkyloxy” is the same as the above “cycloalkyl”.

The aryl part of “arylsulfonyl” or “aryl lower alkyl” is the same as the above “aryl”.

“Heterocyclyl” includes heterocycle which contains at least one hetero atom optionally selected from the group of O, S and N in the ring. Examples include 5- to 6-membered heteroaryl such as pyrrolyl, imidazolyl, pyrazolyl, pyridyl, pyridazinyl, pyrimidinyl, pyrazinyl, triazolyl, triazinyl, tetrazolyl, isoxazolyl, oxazolyl, oxadiazolyl, isothiazolyl, thiazolyl, thiadiazolyl, furyl, thienyl and the like; di-fused heterocyclyl such as indolyl, isoindolyl, indazolyl, indolizinyl, indolinyl, isoindolinyl, quinolyl, isoquinolyl, cinnolinyl, phthalazinyl, quinazolinyl, naphthyridinyl, quinoxalinyl, purinyl, pteridinyl, benzopyranyl, benzimidazolyl, benzisoxazolyl, benzoxazolyl, benzoxadiazolyl, benzisothiazolyl, benzothiazolyl, benzothiadiazolyl, benzofuryl, isobenzofuryl, benzothienyl, benzotriazolyl, imidazopyridyl, triazolopyridyl, imidazothiazolyl, pyrazinopyridazinyl, quinazolinyl, naphthyridinyl, dihydropyridyl, tetrahydroquinolyl, tetrahydrobenzothienyl and the like; tri-fused heterocyclyl such as carbazolyl, acridinyl, xanthenyl, phenothiazinyl, phenoxathiinyl, phenoxazinyl, dibenzofuryl and the like; non-aromatic heterocyclyl such as dioxanyl, thiiranyl, oxiranyl, oxathiolanyl, azetidinyl, thianyl, pyrrolidinyl, pyrrolinyl, imidazolidinyl, imidazolinyl, pyrazolidinyl, pyrazolinyl, piperidyl, piperazinyl, morpholinyl, morpholino, thiomorpholinyl, thiomorpholino, dihydropyridyl, tetrahydrofuryl, tetrahydropyranyl, tetrahydrothiazolyl, tetrahydroisothiazolyl and the like.

“Fused heterocyclyl” which is fused with a ring other than heterocycle (e.g., benzothiazolyl) may have a bonding radical on any ring.

Preferred as heterocyclyl of Z is imidazolyl, benzothiazolyl, isothiazolyl, benzopyranyl, morpholino, pyridyl, quinolyl, pyrimidyl or the like.

Examples of the substituents for “optionally substituted heterocyclyl” are the same as the substituents for the above substituted “cyclic hydrocarbon group”.

The heterocyclyl part of “heterocyclyloxy”, “heterocyclylthio”, “heterocyclylcarbonyl” or “heterocyclylsulfonyl” is the same as the above “heterocyclyl”.

Substituent Group a (alpha) is a group of (1) halogen; (2) oxo; (3) cyano; (4) nitro; (5) imino optionally substituted with lower alkyl or hydroxy; (6) the following groups (i) to (xxi): (i) hydroxy, (ii) lower alkyl, (iii) lower alkenyl, (iv) lower alkoxy, (v) carboxy, (vi) lower alkoxycarbonyl, (vii) acyl, (viii) acyloxy, (ix) imino, (x) mercapto, (xi) lower alkylthio, (xii) carbamoyl, (xiii) lower alkylcarbamoyl, (xiv) cycloalkylcarbamoyl, (xv) thiocarbamoyl, (xvi) lower alkylthiocarbamoyl, (xvii) lower alkylsulfinyl, (xviii) lower alkylsulfonyl, (xix) sulfamoyl, (xx) lower alkylsulfamoyl or (xxi) cycloalkylsulfamoyl, which is optionally substituted with one or more groups selected from Substituent Group β (beta); (7) the following groups (i) to (v): (i) cycloalkyl, cycloalkenyl, (iii) cycloalkyloxy, (iv) amino or (v) alkylene dioxy, which is optionally substituted with (a) substituent(s) selected from Substituent Group β (beta), lower alkyl, lower alkoxy-lower alkyl, optionally protected hydroxyl-lower alkyl, halogeno-lower alkyl, lower alkylsulfonyl and/or arylsulfonyl; and (8) the following groups (i) to (xii):(i) phenyl, (ii) naphthyl, phenoxy, (iv) phenyl-lower-alkoxy, (v) phenylthio, (vi) phenyl-lower-alkylthio, (vii) phenylazo, heterocyclyl, (ix) heterocyclyloxy, (x) heterocyclylthio, (xi) heterocyclylcarbonyl and (xii) heterocyclylsulfonyl, which is optionally substituted with (a) substituent(s) selected from Substituent Group β (beta), lower alkyl, halogeno-lower alkyl and/or oxo.

Substituent Group β (beta) is a group of halogen, optionally protected hydroxy, mercapto, lower alkoxy, lower alkenyl, amino, lower alkylamino, lower alkoxycarbonylamino, lower alkylthio, acyl, carboxy, lower alkoxycarbonyl, carbamoyl, cyano, cycloalkyl, phenyl, phenoxy, lower alkylphenyl, lower alkoxyphenyl, halogenophenyl, naphthyl and heterocyclyl.

Inorganic salt is a salt that consists of the element of alkaline metals (e.g., Li, Na, K, Cs), the element of alkaline-earth metals (e.g., Ca, Ba) or the element of group II (e.g., Mg). Inorganic salt includes sodium salt, lithium salt, potassium salt, magnesium salt, calcium salt, barium salt or a cesium salt.

Preferred is sodium salt, lithium salt or potassium salt.

Organic salt is an ammonium salt that consists of organic amine. Organic amine includes aliphatic amine, cyclic aliphatic amine, aralkylamine, heterocyclic aromatic amine or basic amino acid. Commercial organic amine can be utilized.

Examples of the organic salts include aliphatic amine salt such as trimethylamine salt, triethylamine salt, diisopropylamine salt, dicyclohexylamine salt, ethanolamine salt, diethanolamine salt, triethanolamine salt, procaine salt and the like; cyclic aliphatic amine salt such as N,N-dimethylcyclohexylamine salt, N,N-diethylcyclohexylamine salt, N,N-diisopropylcyclohexylamine salt, N-methylcyclohexylamine salt, N-ethylcyclohexylamine salt, N-isopropylcyclohexylamine salt, cyclohexylamine salt, cyclopentylamine salt, pyrrolidine salt, piperidine salt, piperazine salt, morpholine salt, N-methylmorpholine salt and the like; aralkylamine salt such as N,N-dibenzylethylenediamine salt; heterocyclic aromatic amine salt such as pyridine salt, picoline salt, quinoline salt, isoquinoline salt and the like; quaternary ammonium salt such as tetramethylammonium salt, tetraethylammonium salt, benzyltrimethylammonium salt, benzyltriethylammonium salt, benzyltributylammonium salt, methyltrioctylammonium salt, tetrabutylammonium salt and the like; arginine salt; basic amino acid salt such as lysine salt and the like; and the like.

Preferred is diisopropylamine salt, diisopropylethylamine salt, trimethylamine salt, triethylamine salt, tri-n-propylamine salt, triisopropylamine salt, dimethylethylamine salt, diethylmethylamine salt, diethylisopropylamine salt, dimethylamine salt, methylethylamine salt, diethylamine salt, n-butylamine salt, t-butylamine salt, isobutylamine salt, secondary butylamine salt, isopropylamine salt, n-propylamine salt, ethylamine salt, methylamine salt, N,N-dimethylcyclohexylamine salt, N,N-diethylcyclohexylamine salt, N,N-diisopropylcyclohexylamine salt, N-methylcyclohexylamine salt, N-ethylcyclohexylamine salt, N-isopropylcyclohexylamine salt, cyclohexylamine salt, cyclopentylamine salt, pyrrolidine salt, piperidine salt, piperazine salt, morpholine salt or N-methylmorpholine salt. Especially preferred is pyrrolidine salt, diisopropylamine salt, t-butylamine salt, isopropylamine salt, diisopropylethylamine salt, piperazine salt, piperidine salt, morpholine salt or N-methylmorpholine salt.

More preferred is aliphatic amine salt (e.g., diisopropylamine salt, t-butylamine salt) or cyclic aliphatic amine salt (e.g., pyrrolidine salt).

The salt of the present invention means a salt formed with the carboxyl group of the formula (I). For example, sodium salt means a salt formed with —COO⁻ and Na⁺.

The present compound represented by formula (II) may be the salt thereof. For example, the salt may be the same kind of salt as represented by the formula (I′). Examples of the salts include salt with organic base such as ammonium, trimethylene ammonium, triethyl ammonium or the like; salt with alkali metal such as sodium, potassium or the like; salt with alkaline earth metal such as calcium, magnesium or the like; and the like.

Compounds (I) and (II) may be the solvate thereof which comprises water, acetonitrile, ethyl acetate, methanol, ethanol or the like. The solvation number of the solvate of the present compound, which may usually be changed depending on the synthesis process, the purification method, the crystallization conditions or the like, varies in the range from 0.5 to 5 molecules per molecule of the present compound. The solvate of the salt includes sodium salt 0.5 hydrate, lithium salt monohydrate, potassium salt dihydrate or the like.

Aqueous solution which contains Compounds (I) and (I′) may contain organic solvent.

The present compounds represented by formulae (I), (I′), (II), (IV) and (V) include both of cis-trans isomers. Preferred is trans-isomer. The steric structure can be maintained in the reaction process of the present invention, which is, therefore, a very industrially efficient process.

Compound (IV) can be prepared according to the following processes:

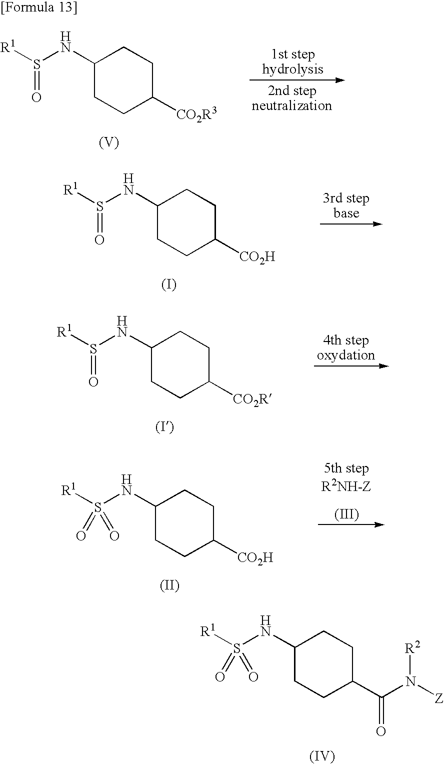

wherein

R¹ and R² have the same meaning as the above,

R³ is optionally substituted lower alkyl, optionally substituted aryl or optionally substituted aryl lower alkyl.

Compound of the formula (I′) is an isolated salt of Compound of the formula (I).

R′ is the element of alkaline metal (e.g., Li, Na, K, Cs), the element of alkaline-earth metal (e.g., Ca, Ba), the element of group II (e.g., Mg) or ammonium salt comprising organic amine.

Compound of the formula (V) may be prepared according to the method disclosed in JP2005-255630.

### 1st Step

1st step is a process for hydrolyzing Compound of the formula (V) in an appropriate solvent in the presence of any base.

This reaction can be performed in a solvent of N-dimethyl formamide, dimethyl sulfoxide, aromatic hydrocarbons (for example, toluene, benzene, xylene or the like), saturated hydrocarbons (for example, cyclohexane, hexane or the like), halogenated hydrocarbons (for example, dichloromethane, chloroform, 1,2-dichloroethane or the like), ethers (for example, tetrahydrofuran, diethyl ether, dioxane, 1,2-dimethoxyethane or the like), esters (for example, methyl acetate, ethyl acetate or the like), ketons (for example, acetone, methyl ethylketone or the like), nitriles (for example, acetonitrile or the like), alcohols (for example, methanol, ethanole, t-butanol or the like), water, a mixed solvent thereof or the like. Preferred is water, alcohols or the mixed solvent thereof.

The amount of the solvent is not specifically limited. The arbitrary amount to give a reactive solution or slurry can be used. For example, when the weight of Compound (V) is v (g), the minimum amount of the solvent can be about 1 v (ml), preferably about 2 v (ml) and more preferably about 3 v (ml). The maximum amount is not specifically limited, but about 10 v (ml), preferably about 8 v (ml) and more preferably about 5 v (ml) considering preparation efficiency. A base is added thereto.

As the base, metal hydroxides (for example, sodium hydroxide, potassium hydroxide, lithium hydroxide, barium hydroxide or the like) can be used. The amount of the base is about 1 mole equivalent or more and preferably about 2 mole equivalents or more, and about 5 mole equivalents or less and preferably about 3 mole equivalents or less relative to 1 mole of Compound (V).

Reaction temperature is not specifically limited, but usually within the range of about 0 to 80° C., preferably about 20 to 50° C.

Reaction time is not specifically limited, but usually within the range of about 1 to 24 hours, preferably about 1 to 10 hours.

At the end of the 1st step, the reaction solution is in the state of an alkaline solution which contains the salt of Compound represented by the formula (I).

### 2nd Step

2nd step is a process for neutralizing by addition of an acid the solution obtained in the 1st step.

As the acid, sulfuric acid, hydrochloric acid, nitric acid, acetic acid, citric acid, oxalic acid or the like can be used. The amount of the acid is not specifically limited. The acid is added until the solution becomes acid, for example, until the pH of the solution reaches 1 to 5.

Reaction temperature is not specifically limited, but usually within the range of about −20 to 40° C. and preferably about 0 to 30° C.

Reaction time is not specifically limited, but usually within the range of about 10 minutes to 2 hours and preferably about 10 minutes to 1 hour.

As shown in Examples described below, Compound of the formula (I) is precipitated as the reaction progresses, which makes it possible to obtain Compound of the formula (I) by filtration after the reaction is completed. As impurities are dissolved in filtrate and removed, the highly-purified product can be obtained in this process.

### 3rd Step

3rd step is a process for preparing a salt of the formula (I′) which comprises dissolving Compound of the formula (I) in an appropriate solvent and adding a base thereto.

As the solvent, the same solvent described in the above 1st step can be used. Preferred is water, but any of the solvents described in the above 1st step can be used as long as it dissolves completely Compound of the formula (I). For example, ethers (for example, tetrahydrofuran, diethyl ether, dioxane, 1,2-dimethoxyethane or the like) can be used, and ether-water mixed solvents can also be used.

As the base, metal hydroxides (for example, sodium hydroxide, potassium hydroxide, lithium hydroxide, barium hydroxide or the like) or organic amines can be used. The amount of the base is about 0.9 to 1 mole equivalent relative to 1 mole of Compound (I). More than 1 mole equivalent of the base may be used, and in that case, since there is a possibility of the excess base deactivating an oxidizer which is used in the following process, it is necessary to add the oxidizer as appropriate until the reaction is completed in the following process. In the case of less than 1 mole equivalent of the base, the unreacted Compound (I) may be removed by filtration and the like.

Reaction temperature is not specifically limited, but usually within the range of about −30 to 40° C. and preferably within the range of about −20 to 30° C.

Reaction time is not specifically limited, but usually within the range of about 10 minutes to 2 hours and preferably within the range of about 10 minutes to 1 hour.

When Compound of the formula (I) is insoluble in the solvent, it is difficult to complete the reaction in this process and thus, it is preferable to perform this process after Compound of the formula (I) is dissolved completely in the solvent.

### 4th Step

4th step is a process for dissolving Compound of the formula (I′) in an appropriate solvent and oxidizing thereof.

As the solvent, the same solvent described in the above 1st step can be used. Preferred is water, but any of the solvents described in the above 1st step can be used as long as it dissolves completely Compound of the formula (I).

An arbitrary oxidizer can be used. Examples of the oxidizers include peracetic acid, performic acid, m-chloroperbenzoic acid, pertrifluoroacetic acid, sodium periodate, magnesium monoperoxy phthalate (MMPP), potassium permanganate, sodium hypochlorite, calcium hypochlorite, perchloric acid, chlorous acid, oxone (2KHSO₅.KHSO₄.K₂SO₄), O₂ and the like. Preferred is hydrogen peroxide.

Hydrogen peroxide can be used as hydrogen peroxide solution. As a catalyst, ammonium molybdate tetrahydrate ((NH₄)₆Mo₇ O_{2 4}.4H₂O), sodium tungstate, the hydrate thereof or the like can be used. The amount of peroxide is about 1 mole equivalent or more, and about 3 mole equivalents or less and preferably 2 mole equivalents or less relative to 1 mole of Compound (I′). The minimum amount of catalyst is about 0.005 mole equivalent or more and preferably about 0.01 mole equivalent or more, and about 0.1 mole equivalent or less and preferably about 0.06 mole equivalent or less relative to 1 mole of Compound (I′).

Reaction temperature is not specifically limited, but usually within the range of about 0 to 100° C. and preferably within the range of about 20 to 60° C.

Reaction time is not specifically limited, but usually within the range of about 1 to 24 hours and preferably within the range of about 1 to 5 hours.

After the reaction is completed, Compound (II) is crystallized by adding thereto an acid such as sulfuric acid, hydrochloric acid or the like within the range of about 10 to 50° C., preferably within the range of about 20 to 30° C., and stirring for about 15 minutes to 10 hours, preferably about 30 minutes to 3 hours. Then, washing, filtrating and drying by well-known methods affords Compound (II).

In the 4th step, the pH of the solution that contains Compound of the formula (I′) is critical. Preferred is the pH between 6 and 11. In the case that the solution is more acidic than the pH 6, Compound of the formula (I) is not dissolved in water, a precipitate is formed, and as a result, the oxidation reaction is inhibited. In the case that the solution is more alkaline than the pH 10, the oxidation reagent is decomposed and the oxidation reaction is inhibited. Therefore, it is essential to maintain the pH of the solution between 6 and 11, to keep the equilibrium conditions of Compound of the formula (I) and Compound of the formula (I′) in the solution and then, to carry out the oxidation reaction. The preferred pH of the solution is between 6 and 8, more preferred is between 6.6 and 7.4, and especially preferred is between 7.3 and 7.4.

### 5th Step

5th step is a process for preparing Compound of the formula (IV) which comprises reacting Compound of the formula (II) with Compound of the formula (III).

This reaction can be carried out according to the amidation reaction disclosed in the above Patent Document 1 or the like.

For example, Compound (III) and an activator such as acid halide (for example, thionyl chloride, oxalyl chloride, phosphorus oxychloride or the like is used), acid anhydride or activated ester of Compound (II) are reacted in an appropriate solvent within the range of about 0 to 100° C. for about 3 minutes to 10 hours.

As the solvent, the same solvent described in the above 1st step can be used. Tetrahydrofuran, dimethylformamide, diethyl ether, dichloromethane, toluene, benzene, xylene, cyclohexane, hexane, chloroform, ethyl acetate, butyl acetate, pentane, heptane, dioxane, acetone, acetonitrile, water, the mixed solvent thereof or the like can be used. Preferred is toluene or tetrahydrofuran. Additionally, if necessary, an activator such as base (preferred is triethylamine, pyridine or the like), thionyl chloride, acid halide (e.g., thionyl chloride, oxalyl chloride, phosphorus oxychloride or the like), acid anhydride or activated ester can be used.

As an alternative process, Compound (IV) can be obtained by reacting Compound (II) and Compound (III) in an appropriate solvent (e.g., tetrahydrofuran, dimethylformamide, diethyl ether, dichloromethane, toluene, benzene, xylene, cyclohexane, hexane, chloroform, ethyl acetate, butyl acetate, pentane, heptane, dioxane, acetone, acetonitrile, water, the mixed solvent thereof or the like) in the presence of a condensing agent within the range of about 0 to 100° C. for about 3 minutes to 10 hours.

Examples of the condensing agents include 1,1-carbonyldiimidazole, dicyclohexyl carbodiimide, water soluble carbodiimide (1-ethyl-3-(3′-dimethylamino propyl)carbodiimide) and the like.

Examples of the groups of Z include the following:

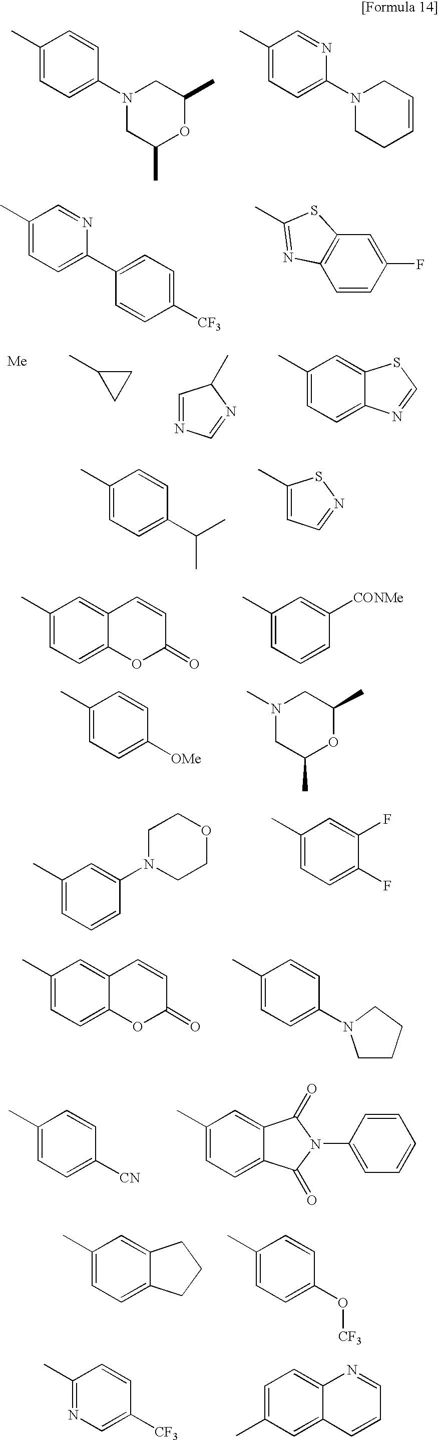

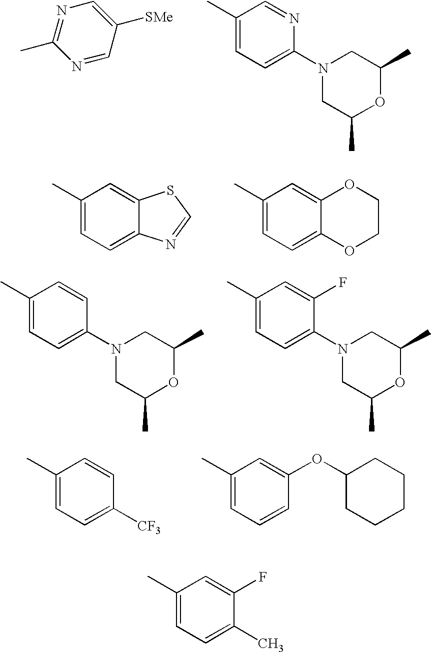

This process can be performed according to the methods disclosed in WO2003/076374. For example, this process can be performed in the same methods as described in Examples 8 to 12 in WO2003/076374.

Compound (IV) obtained as above is useful as an NPY Y5 receptor antagonist.

This invention is further explained by the following Examples, which are not intended to limit the scope of the present invention.

### Reference Example 1

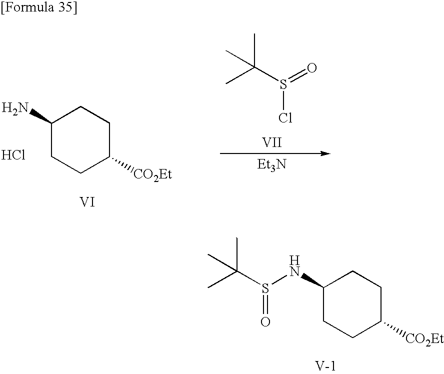

To hydrochloride of Compound (VI) (130.00 g) were added toluene (390 mL), tap water (260 mL) and triethylamine (139.34 g) at the temperature of 15° C. A solution of Compound (VII) (95.86 g) dissolved in toluene (130 mL) was added dropwise thereto at the temperature from 10 to 20° C. over 28 minutes. This reaction solution was stirred at the temperature from 10 to 20° C. for about 60 minutes and then separated to obtain the upper layer (628.66 g) (Compound (V-1) in toluene solution).

### Example 1

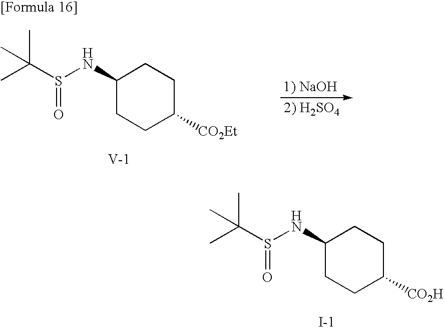

To this reaction solution obtained in Reference Example 1 were added tap water (403 mL) and 48% NaOH solution (130.38 g). The reaction mixture was stirred at the temperature about 40° C. for 2 hours, and then separated to obtain the lower layer (695.12 g). To this reaction solution (413.13 g) was added tap water (60 mL), and 20% sulfuric acid solution (258.17 g) was added dropwise thereto at the temperature from 5 to 25° C. to be pH 2. This reaction mixture was stirred at the temperature about 10° C. for 20 minutes and then filtered. The residue was washed with tap water (250 mL). The undried crystal was collected and air-dried at room temperature to give 89.07 g of Compound (1-1) (96.8% yield, based on hydrochloride of Compound (VI)).

### Example 2

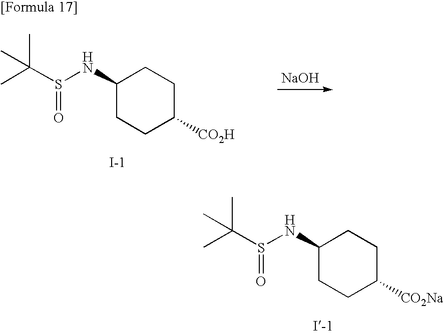

10.0 g of Compound (1-1) obtained in Example 1 was dissolved by adding tap water (70 mL) and NaOH (1.70 g). The resulting solution was filtered and distilled away under reduced pressure to give Compound (I′-1).

### Elemental Analysis:

Calcd C, 47.47%; H, 7.60%; N, 5.03%; S, 11.52%; Na, 8.26%.

Found: C, 47.05%; H, 7.62%; N, 5.10%; S, 11.07%; Na, 8.18%. (0.5H₂O)

¹H-NMR DMSO (internal standard: TMS) 300 MHz δ1.04-1.31 (4H, m), 1.09 (9H, s), 1.56-1.70 (1H, m), 1.74-1.95 (4H, m), 2.76-2.93 (1H, m), 4.84 (1H, d, J=6.3 Hz)

Melting point: Degraded over 175° C.

### Example 3

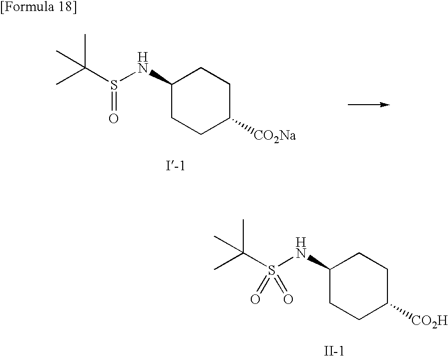

To 5.98 g of Compound (I′-1) obtained in Example 2 was added tap water (21.0 mL) to be a solution of pH between 7.3 and 7.4. To this reaction solution was added sodium tungstate dihydrate (79.5 mg). 35% hydrogen peroxide solution (4.68 g) was added dropwise thereto at the temperature from 25 to 35° C. over 25 minutes. This reaction solution was stirred at the temperature about 25° C. for 8 hours. 31.49 g of a solution of sodium sulfite (7.00 g) dissolved in tap water (93 g) was added dropwise thereto and the excess peroxide was removed. To this reaction solution was added dropwise 20% sulfuric acid solution (7.85 g) at the temperature from 25 to 35° C. to be pH 2. This reaction mixture was stirred at the temperature about 25° C. for 30 minutes, left as it was all night long, filtered, and then the residue was washed with 30 mL of tap water. The undried crystal was collected and dried under reduced pressure with heating (85° C.) to give 5.60 g of Compound (II-1) (95.8% yield, based on sodium salt of Compound (I′-1)).

### Example 4

In the same methods as described in Example 2, Compound (I′-1) was prepared by using different bases in place of NaOH.

### (Salt: Lithium Salt)

### Elemental Analysis:

Calcd: C, 48.70%; H, 8.17%; N, 5.16%; S, 11.82%; Li, 2.56%.

Found: C, 48.57%; H, 8.16%; N, 5.24%; S, 12.05%; Li, 2.46%. (1.0H₂O).

¹H-NMR: DMSO (internal standard: TMS) 300 MHz δ1.03-1.33 (4H, m), 1.08 (9H, s), 1.61-1.75 (1H, m), 1.76-1.95 (4H, m), 2.74-2.93 (1H, m), 4.89 (1, d, J=6.6 Hz)

Melting point: Degraded over 233° C.

### (Salt: Potassium Salt)

### Elemental Analysis:

Calcd: C, 41.10%; H, 7.52%; N, 4.36%; S, 9.98%; K, 12.16%.

Found: C, 40.89%; H, 7.51%; N, 4.45%; S, 10.28%; K, 12.03%. (2.0H₂O)

¹H-NMR: DMSO (internal standard: TMS) 300 MHz δ1.04-1.31 (4H, m), 1.09 (9H, s), 1.53-1.68 (1H, m), 1.73-1.93 (4H, m), 2.74-2.91 (1H, m), 4.87 (1H, d, J=6.6 Hz)

Melting point: Degraded over 185° C.

### (Salt: Diisopropylamine Salt)

### Elemental Analysis:

Calcd: C, 58.58%; H, 10.41%; N, 8.04%; S, 9.20%.

Found: C, 57.90%; H, 10.52%; N, 8.00%; S, 9.28%.

¹H-NMR: DMSO (internal standard: TMS) 300 MHz δ0.99 (12H, d, J=6.0 Hz), 1.09 (9H, s), 1.14-1.43 (4H, m), 1.75-2.10 (5H, m), 2.80-2.99 (3H, m), 4.95 (1H, d, J=6.6 Hz)

Melting point: Degraded over 200° C.

### (Salt: Pyrrolidine Salt)

### Elemental Analysis:

Calcd: C, 56.57%; H, 9.49%; N, 8.80%; S, 10.07%.

Found: C, 56.23%; H, 9.39%; N, 8.72%; S, 10.07%.

¹H-NMR: DMSO (internal standard: TMS) 300 MHz δ1.09 (9H, s), 1.14-1.37 (4H, m), 1.64-1.74 (4H, m), 1.79-1.98 (5H, m), 2.79-2.95 (5H, m), 4.95 (1H, d, J=6.6 Hz)

Melting point: 145.0-145.4° C.

### (Salt: T-Butyl Amine Salt)

### Elemental Analysis:

Calcd: C, 56.21%; H, 10.06%; N, 8.74%; S, 10.00%.

Found: C, 55.61%; H, 9.88%; N, 8.59%; S, 9.87%.

¹H-NMR:DMSO (internal standard: TMS) 300 MHz δ1.04-1.38 (4H, m), 1.08 (9H, s), 1.12 (9H, s), 1.77-1.99 (5H, m), 2.80-2.97 (1H, m), 4.95 (1H, d, J=6.6 Hz)

Melting point: Degraded over 174° C.

When the above salts dissolve in water, the pH of the resulting solutions is between 6 and 11.

### Example 5

Compound (I-1) was dissolved by adding tap water and sodium hydroxide. To the resulting solution was added hydrochloric acid dropwise to adjust the pH thereof to between 6 and 11.

### Example 6

Compound (I-1) was dissolved by adding tap water and potassium hydroxide. To the resulting solution was added hydrochloric acid dropwise to adjust the pH thereof to between 6 and 11.

### Example 7

Compound (I-1) was dissolved by adding THF and t-butyl amine. To the resulting solution was added hydrochloric acid dropwise to adjust the pH thereof to between 6 and 11.

### Example 8

The solutions of pH between 6 and 11 obtained in Examples 5 to 7 were oxidized according to the process of Example 3 to give Compound (II-1).

### INDUSTRIAL APPLICABILITY

Processes of the present invention enable Compounds (I) and (II) to be prepared safely and efficiently, and thus, they are useful as an industrial preparation method.

1. A salt or a solvate thereof, of a compound represented by formula (I): 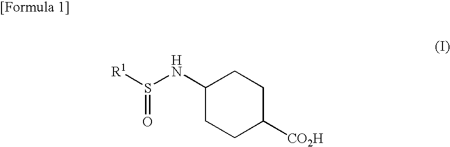
wherein R¹ is optionally substituted lower alkyl, optionally substituted cycloalkyl or optionally substituted aryl.

2. The salt or a solvate thereof, of the compound according to claim 1, wherein the salt is an inorganic salt.

3. The salt or a solvate thereof, of the compound according to claim 1, wherein the salt is selected from a group consisting of sodium salt, lithium salt, potassium salt, magnesium salt, calcium salt, barium salt and cesium salt.

4. The salt or a solvate thereof, of the compound according to claim 1, wherein the salt is an organic salt.

5. The salt or a solvate thereof, of the compound according to claim 4, wherein the salt is selected from a group consisting of pyrrolidine salt, diisopropylamine salt, tert-butylamine salt, isopropylamine salt, diisopropylethylamine salt, piperazine salt, piperidine salt, morpholine salt and N-methyl morpholine salt.

6. An aqueous solution of pH between 6 and 11 containing a compound represented by formula (I): 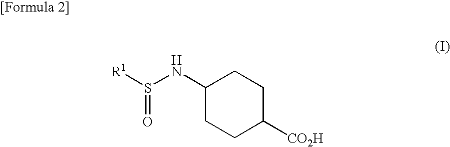
wherein R¹ has the same meaning as defined in claim 1, or the salt thereof.

7. A process for the preparation of an aqueous solution of pH between 6 and 11 containing a compound represented by formula (I): 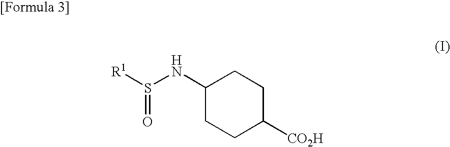
wherein R¹ has the same meaning as defined in claim 1, or the salt thereof,
which comprises neutralizing with an acid an aqueous solution containing a salt of the compound represented by the formula (I).

8. A process for the preparation of Compound (II): 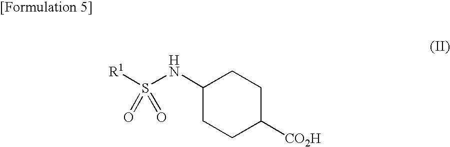
wherein R¹ is optionally substituted lower alkyl, optionally substituted cycloalkyl or optionally substituted aryl,
which comprises oxidizing an aqueous solution of pH between 6 and 11 containing a compound represented by the formula (I): 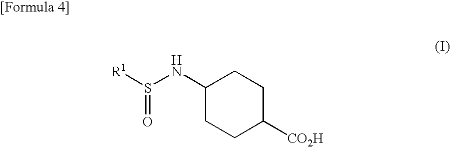
wherein R¹ has the same meaning as defined above, or a salt thereof.

9. A process for the preparation of a salt or its solvate, of a compound represented by the formula (I): 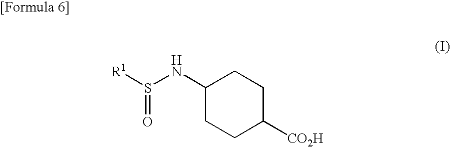
wherein R¹ has the same meaning as defined in claim 1,
which comprises dissolving or suspending a compound represented by the formula (I) in water and/or organic solvent and adding a base thereto.

10. A compound represented by the formula (IV): 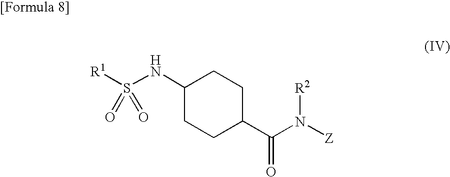
wherein R¹ is optionally substituted lower alkyl, optionally substituted cycloalkyl or optionally substituted aryl, R² is hydrogen or lower alkyl and Z is optionally substituted lower alkyl, optionally substituted lower alkenyl, optionally substituted amino, optionally substituted lower alkoxy, optionally substituted cyclic hydrocarbon group or optionally substituted heterocyclic group,
its pharmaceutically acceptable salt or solvate thereof,
which comprises reacting a Compound of formula (II): 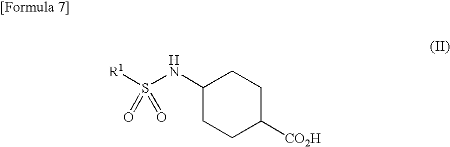
wherein R¹ has the same meaning as defined above,
with a Compound represented by formula (III): R²NH—Z (H),
wherein R² and Z have the same meaning as defined above for formula (IV).

11. An aqueous solution of the pH between 6.6 and 7.4 containing a compound represented by the formula (I): 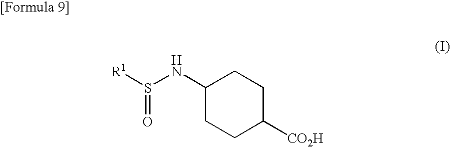
wherein R¹ has the same meaning as defined in claim 1,
or the salt thereof.
